(19) United States
(12) Patent Application Publication
(10) Pub. No.: US 2022/0125751 A1
(43) Pub. Date: Apr. 28, 2022

(54) NOVEL COMPOSITION

(71) Applicant: Reckitt Benckiser Health Limited, Berkshire (GB)

(72) Inventors: Fraser William Hanson BROWN, Humberside (GB); Steven Scott HALL, Humberside (GB); Rouzbeh MIRFATTAHI, Humberside (GB); Delphine Bérengère SON, Humberside (GB)

(73) Assignee: Reckitt Benckiser Health Limited, Berkshire (GB)

(21) Appl. No.: 17/429,761

(22) PCT Filed: Feb. 11, 2020
(86) PCT No.: PCT/GB2020/050316

§ 371 Date: Aug. 10, 2021

(30) Foreign Application Priority Data
Feb. 11, 2019 (GB) 1901876.1
Feb. 19, 2019 (GB) 1902257.3

(51) Int. Cl. A61K 31/192; A61K 31/196; A61K 47/38; A61K 47/40; A61K 47/02; A61K 47/12; A61K 47/36; A61K 9/00

(52) U.S. Cl. CPC A61K 31/192; A61K 31/196; A61K 47/38; A61K 9/006; A61K 47/02; A61K 47/12; A61K 47/36; A61K 47/40

(57) ABSTRACT

A composition comprising a pharmaceutically active ingredient, a solubilising agent and a thixotropic agent.

The present invention is directed to a pharmaceutical composition in the form of an aqueous spray suitable for the administration of a pharmaceutical active to the throat/pharyngeal area. In particular, the present invention is directed to a thixotropic composition for administration of an NSAID.

Spray compositions are known for application to different surfaces of the body, such as nasal sprays, breath fresheners and antiseptic sprays for skin application.

A common problem with spray administration is a low efficiency of the active agent, since the structure of body cavities and parts does not typically facilitate retention of the applied formulation. This is particularly the case for aqueous-based spray formulations, which must have sufficient fluidity to be dispensed by a pump device or a squeeze-type spray bottle, but which can simply drain from the area of application while, or immediately after, being sprayed. Due to swallowing of much of the formulation which enters the oropharyngeal area, a large portion of the active agent introduced into the mouth and/or nose is generally rendered unavailable for its intended use.

It would be desirable to provide an aqueous composition that can be made to have a viscosity sufficiently low to permit spraying with a standard pump mechanism or squeeze-type spray bottle, but which then rapidly exhibits a significant viscosity increase to retain the composition at the application site.

Thixotropic compositions exhibit a time-dependent shear thinning property—under static conditions the compositions are thick or viscous but become thin and less viscous when shaken or agitated. The effect of the shaking or agitation is to cause the particles/polymers in the composition to temporarily shear as a result of the stress placed on them by the shaking or agitation.

However, the materials/agents which are used to impart thixotropic properties to a composition are typically polymeric materials. The inclusion of such polymeric materials can impact on the ability of the composition to solubilise certain pharmaceutically active compounds. This is because salt buffers are generally required to solubilise pharmaceutically active compounds. However, the salt buffers interfere with the polymer matrix and the gel structure is disrupted. As a result the active becomes insoluble and flocculation/syneresis occurs.

In addition, thixotropic spray compositions which are suitable for application to the nasal cavity are not suitable for the administration of a pharmaceutically active ingredient to the throat/pharyngeal area as a result of the spray pattern of such compositions. Nasal sprays form a wide spray angle on administration to ensure that a large surface area of the nasal passage is coated with the spray. In contrast, a thixotropic or shear-thinning throat spray should have a narrow spray angle to ensure that the composition coats only the surface of the throat/pharyngeal area and not the entire oral cavity.

Indeed, there are no thixotropic compositions currently available which are suitable for the administration of a pharmaceutically active ingredient to the mucosal tissue of the throat/pharyngeal area.

There exists a need to provide a thixotropic composition which comprises a pharmaceutically active ingredient in solution and has the required rheological properties.

The present invention provides an aqueous-based sprayable composition containing a pharmaceutically active ingredient, and a thixotropic material. The composition is a non-Newtonian thixotropic fluid, exhibiting a reduced apparent viscosity while being subjected to shear forces, but a high apparent viscosity while at rest; this property permits application by spraying with readily available pump spray devices or squeeze-type spray bottles immediately following the application of a shearing force (such as those created by vigorously shaking the product container), but causes the sprayed material to remain at least temporarily relatively immobile on mucosal membranes or the skin.

According to an aspect of the present invention there is provided a composition comprising a pharmaceutically active ingredient, a solubilising agent and a thixotropic agent.

Preferably, the composition is in the form of a solution.

Typically, the composition is in the form of a sprayable composition.

The pharmaceutically active ingredient can be selected from NSAIDs or pharmaceutically acceptable salts thereof. A preferred NSAID is taken from the group consisting of flurbiprofen, ketoprofen or diclofenac. A more preferred NSAID is flurbiprofen.

Other pharmaceutically active ingredients that can be used are hexylresorcinol, benzocaine, dextromethorphan, menthol lidocaine, amyl metacresol and 2,4-dichlorobenzyl alcohol or pharmaceutically acceptable salts thereof.

The composition can comprise up to about 5% NSAID by weight of the composition. Typically, the amount of NSAID is from 0.5% by weight of the composition to 3% by weight of the composition. More typically, the amount of NSAID is from 1% by weight of the composition to 2% by weight of the composition.

The composition can comprise 10-25 mg/ml of NSAID. Preferably the composition comprises 15-18 mg/ml of NSAID. More preferably the composition comprises 16-17 mg/ml of NSAID.

When the active is selected to be hexylrescinol the composition can comprise 2-5 mg/ml of the active. When the active is selected to be benzocaine the composition can comprise 10-20 mg/ml. When the active is selected to be dextromethorphan the composition can comprise 15-30 mg/ml. When active is selected to be menthol the composition can comprise 5-15 mg/ml. When the active is selected to be lidocaine the composition can comprise 2-5 mg/ml. When the active is selected to be amyl metacresol the composition can comprise 0.5-1.5 mg/ml. When the active is selected to be 2,4-dichlorobenzyl alcohol the composition can comprise 1-2.5 mg/ml.

Preferably, the composition has a very rapid rate of viscosity recovery, following withdrawal of the shearing force. A ‘very rapid rate of viscosity recovery’ means that the viscosity of the composition returns to about 90% of its initial viscosity within 30 s following withdrawal of the shearing force. More preferably, the viscosity of the composition returns to about 90% of its initial viscosity within 10 s following withdrawal of the shearing force. Most preferably, the viscosity of the composition returns to about 90% of its initial viscosity within 5 s following withdrawal of the shearing force. As used herein the term ‘initial viscosity’ is the viscosity of the composition prior to the application of a shear force.

The thixotropic agent can be present in an amount of 0.1-15% by weight of the composition. Preferably, the thixotropic agent can be present in an amount of 0.5-10% by weight of the composition. More preferably, the thixotropic agent can be present in an amount of 0.5-8% by weight of the composition. More preferably, thixotropic agent can be present in an amount of 1-5% by weight of the composition.

The thixotropic agent can be selected from combinations of one or more celluloses and one or more alkali metal carboxyalkylcelluloses, carrageenan and xanthan gum. Preferably, the thixotropic agent is selected from a combination of one or more celluloses and one or more alkali metal carboxyalkylcelluloses.

A preferred combination of cellulose and alkali metal carboxyalkylcellulose is microcrystalline cellulose and sodium carboxymethylcellulose. Typically this combination contains 85-95 weight percent microcrystalline cellulose and 5-15 weight percent sodium carboxymethylcellulose. More typically this combination contains 86-92 weight percent microcrystalline cellulose and 8-14 weight percent sodium carboxymethylcellulose.

Typically, the compositions of the present invention contain at least about 1.5 weight percent of the cellulose/carboxyalkylcellulose combination up to about 10 weight percent to avoid producing high viscosities which impede spraying with the usual devices. Preferably, the compositions contain about 1.5 to about 5 weight percent of the mixture. More preferably, the amount will be about 2.0 to about 3.0 weight percent. Most preferably, the composition contains about 2.2 to about 2.8 weight percent.

The solubilising agent can be present in the composition in an amount of 0.1-15 w/w %. Typically, the solubilising agent can be present in an amount of 1-10 w/w %. Typically, the solubilising agent can be present in the composition in an amount of 3-7 w/w %. Typically, the solubilising agent can be present in an amount of 5-7 w/w %. More typically, the solubilising agent can be present in an amount of 6 w/w %.

The solubilising agent is typically selected from cyclodextrins, or combinations thereof. Other solubilising agents include non-ionic surfactants, such as Tween, and propylene glycol.

The cyclodextrin can be selected from α, β, γ cyclodextrin and derivatives thereof. Cyclodextrins for use in the present invention include the natural cyclodextrins and their derivatives, including the alkylated and hydroxyalkylated derivatives and the branched cyclodextrins. derivatives bearing sugar residues are of special interest. Especially useful herein are the hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α, β, γ-cyclodextrin. Specific cyclodextrin derivatives for use herein include α-cyclodextrin, β-cyclodextrin, γ-cyclodextrin, methyl-α-cyclodextrin, hydroxyethyl-α-cyclodextrin, hydroxypropyl-α-cyclodextrin, dihydroxypropyl-α-cyclodextrin, methyl-β-cyclodextrin, hydroxyethyl-β-cyclodextrin, hydroxypropyl-β-cyclodextrin, dihydroxypropyl-β-cyclodextrin, methyl-γ-cyclodextrin, hydroxyethyl-γ-cyclodextrin, hydroxypropyl-γ-cyclodextrin and dihydroxypropyl-γ-cyclodextrin.

Preferably, the cyclodextrin comprises more than one cyclodextrin. More preferably the cyclodextrin comprises 2 cyclodextrins.

Typically, the molar ratio of cyclodextrins is from 1:1 to 1:2 by weight percent of the composition. Preferably, the molar ratio of cyclodextrins is 1:1.9.

Alternatively the molar ratio of the cyclodextrins is 1:2.

The cyclodextrin can comprise a combination of β-cyclodextrin and hydroxypropyl-β-cyclodextrin.

The composition can comprise one or more stabilisers. The one or more stabilisers can be selected from ethyl cellulose, hydroxypropyl methyl cellulose and xanthan gum. A preferred stabiliser is xanthan gum.

The one or more stabilisers can be present at an amount of 0.05-5% w/w. Preferably the amount of the one or more stabilisers is 0.1-1% w/w. More preferably the amount of the stabilisers is 0.15-0.5% w/w.

The pH of the composition is preferably from 6.0-8.0. More preferably from 7.0-7.6. Most preferably the pH is about 7.4.

The composition can further include one or more buffers. The one or more buffers is selected to ensure that the pH of the composition is between about 6.0 and 8.0. Preferably the one or more buffers is selected to ensure that the pH of the composition is between 7.0 and 7.6. More preferably the one or more buffers is selected to ensure that the pH of the composition is about 7.4.

The one or more buffers can be present at an amount of 0.1-15% w/w. Preferably the amount of the one or more buffers is 1-5% w/w. More preferably the amount of the buffers is 2-4% w/w.

Preferably, the one or more buffers is selected from citric acid, disodium hydrogen phosphate, sodium hydrogen phosphate and combinations thereof.

Typically, the one or more buffers comprises citric acid and disodium hydrogen phosphate. More typically, the composition comprises a combination of citric acid and disodium hydrogen phosphate.

Preferably, the composition comprises from 0.1-5% w/w of citric acid and 1-10% w/w disodium hydrogen phosphate. More preferably, the composition comprises from 0.1-0.5% w/w of citric acid and 1-5% w/w disodium hydrogen phosphate.

The composition can further include additional pharmaceutically acceptable excipients selected from sweeteners, aesthetic agents, flavours, preservatives, pH adjusters and non-ionic buffers.

The composition can comprise:

| | | |
|---|---|---|
| (a) | 1-5% | An NSAID; |
| (b) | 0.1-15% | A thixotropic agent; and |
| (c) | 0.1-15% | One or more solubilisers. |

The composition can comprise:

| | | |
|---|---|---|
| (a) | 1-5% | An NSAID; |
| (b) | 1-5% | A thixotropic agent; |
| (c) | 1-15% | One or more solubilisers; |
| (d) | 0.1-10% | One or more pH adjusters; and |
| (e) | 0.1-15% | One or more buffers. |

The composition can comprise:

| | | |
|---|---|---|
| (a) | 1-5% | An NSAID; |
| (b) | 1-5% | A thixotropic agent; |
| (c) | 1-15% | One or more solubilisers; |
| (d) | 1-10% | One or more pH adjusters; and |
| (e) | 0.1-15% | One or more buffers. |

The composition can comprise:

| | | |
|---|---|---|
| (a) | 1-5% | An NSAID; |
| (b) | 0.5-8% | A thixotropic agent; |
| (c) | 1-10% | One or more solubilisers; |
| (d) | 0.1-1% | One or more pH adjusters; and |
| (e) | 1-5% | One or more buffers. |

The composition can comprise:

| | | |
|---|---|---|
| (a) | 1-5% | An NSAID; |
| (b) | 0.5-8% | A thixotropic agent; |
| (c) | 1-7% | One or more solubilisers; |
| (d) | 0.1-1% | One or more pH adjusters; and |
| (e) | 1-5% | One or more buffers. |

The composition can comprise:

| | | |
|---|---|---|
| (a) | 1-5% | An NSAID; |
| (b) | 1-5% | A thixotropic agent; |
| (c) | 1-7% | One or more solubilisers; |
| (d) | 0.1-1% | One or more pH adjusters; and |
| (e) | 1-5% | One or more buffers. |

The composition can comprise:

| | | |
|---|---|---|
| (a) | 1-5% | An NSAID; |
| (b) | 1-5% | A thixotropic agent; |
| (c) | 1-15% | One or more solubilisers; |
| (d) | 0.1-15% | One or more buffers; and |
| (e) | 0.05-5% | One or more stabilisers. |

The composition can comprise:

| | | |
|---|---|---|
| (a) | 1-5% | An NSAID; |
| (b) | 0.5-8% | A thixotropic agent; |
| (c) | 1-7% | One or more solubilisers; |
| (d) | 1-5% | One or more buffers; and |
| (e) | 0.05-5% | One or more stabilisers. |

The composition can comprise:

| | | |
|---|---|---|
| (a) | 1-5% | An NSAID; |
| (b) | 1-5% | A thixotropic agent; |
| (c) | 1-7% | One or more solubilisers; |
| (d) | 1-5% | One or more buffers; and |
| (e) | 0.05-5% | One or more stabilisers. |

The composition can comprise:

| | | |
|---|---|---|
| (a) | 1-5% | An NSAID; |
| (b) | 1-5% | A thixotropic agent; |
| (c) | 1-15% | One or more solubilisers; |
| (d) | 0.1-10% | One or more pH adjusters; |
| (e) | 0.1-15% | One or more buffers; and |
| (f) | 0.05-5% | One or more stabilisers. |

The composition can comprise:

| | | |
|---|---|---|
| (a) | 1-5% | An NSAID; |
| (b) | 1-5% | A thixotropic agent; |
| (c) | 1-15% | One or more solubilisers; |
| (d) | 1-10% | One or more pH adjusters; |
| (e) | 0.1-15% | One or more buffers; and |
| (f) | 0.05-5% | One or more stabilisers. |

The composition can comprise:

| | | |
|---|---|---|
| (a) | 1-5% | An NSAID; |
| (b) | 0.5-8% | A thixotropic agent; |
| (c) | 1-10% | One or more solubilisers; |
| (d) | 0.1-1% | One or more pH adjusters; |
| (e) | 1-5% | One or more buffers; and |
| (f) | 0.1-1% | One or more stabilisers. |

The composition can comprise:

| | | |
|---|---|---|
| (a) | 1-5% | An NSAID; |
| (b) | 0.5-8% | A thixotropic agent; |
| (c) | 1-7% | One or more solubilisers; |
| (d) | 0.1-1% | One or more pH adjusters; |
| (e) | 1-5% | One or more buffers; and |
| (f) | 0.1-1% | One or more stabilisers. |

The composition can comprise:

| | | |
|---|---|---|
| (a) | 1-5% | An NSAID; |
| (b) | 1-5% | A thixotropic agent; |
| (c) | 1-7% | One or more solubilisers; |
| (d) | 0.1-1% | One or more pH adjusters; |
| (e) | 1-5% | One or more buffers; and |
| (f) | 0.1-1% | One or more stabilisers. |

The composition can comprise

| | | |
|---|---|---|
| (a) | 1-5% | An NSAID; |
| (b) | 0.1-15% | A combination of a cellulose and an |
| | | alkali metal carboxyalkylcellulose; and |
| (c) | 0.1-15% | One or more cyclodextrins. |

The composition can comprise:

| | | |
|---|---|---|
| (a) | 1-2% | Flurbiprofen; |
| (b) | 2-4% | A combination of a cellulose and an |
| | | alkali metal carboxyalkylcellulose; and |
| (c) | 5-7% | One or more cyclodextrins. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-15% | One or more cyclodextrins; |
| (d) | 0.1-10% | One or more pH adjusters; and |
| (e) | 0.1-15% | One or more buffers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-15% | One or more cyclodextrins; |
| (d) | 1-10% | One or more pH adjusters; and |
| (e) | 0.1-15% | One or more buffers. |

The composition can comprise:

| | | |
|---|---|---|
| (a) | 1-5% | An NSAID; |
| (b) | 0.5-8% | A combination of a cellulose and an alkali metal |
| | | carboxyalkylcellulose; |
| (c) | 1-10% | One or more cyclodextrins; |
| (d) | 0.1-1% | One or more pH adjusters; and |
| (e) | 1-5% | One or more buffers. |

The composition can comprise:

| | | |
|---|---|---|
| (a) | 1-5% | An NSAID; |
| (b) | 0.5-8% | A combination of a cellulose and an alkali metal |
| | | carboxyalkylcellulose; |
| (c) | 1-7% | One or more cyclodextrins; |
| (d) | 0.1-1% | One or more pH adjusters; and |
| (e) | 1-5% | One or more buffers. |

The composition can comprise:

| | | |
|---|---|---|
| (a) | 1-2% | Flurbiprofen; |
| (b) | 2-4% | A combination of a cellulose and an alkali metal |
| | | carboxyalkylcellulose; |
| (c) | 5-7% | One or more cyclodextrins; |
| (d) | 0.1-0.5% | One or more pH adjusters; and |
| (e) | 2-4% | One or more buffers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-7% | One or more cyclodextrins; |
| (d) | 0.1-1% | One or more pH adjusters; and |
| (e) | 1-5% | One or more buffers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-15% | One or more cyclodextrins; |
| (d) | 0.1-15% | One or more buffers; and |
| (e) | 0.05-5% | One or more stabilizers. |

The composition can comprise:

| | | |
|---|---|---|
| (a) | 1-5% | An NSAID; |
| (b) | 0.5-8% | A combination of a cellulose and an alkali metal |
| | | carboxyalkylcellulose; |
| (c) | 1-7% | One or more cyclodextrins; |
| (d) | 1-5% | One or more buffers; and |
| (e) | 0.1-1% | One or more stabilizers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-7% | One or more cyclodextrins; |
| (d) | 1-5% | One or more buffers; and |
| (e) | 0.1-1% | One or more stabilizers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-15% | One or more cyclodextrins; |
| (d) | 0.1-10% | One or more pH adjusters; |
| (e) | 0.1-15% | One or more buffers; and |
| (f) | 0.05-5% | One or more stabilizers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-15% | One or more cyclodextrins; |
| (d) | 1-10% | One or more pH adjusters; |
| (e) | 0.1-15% | One or more buffers; and |
| (f) | 0.05-5% | One or more stabilizers. |

The composition can comprise:

| | | |
|---|---|---|
| (a) | 1-2% | Flurbiprofen; |
| (b) | 2-4% | A combination of a cellulose and an alkali metal |
| | | carboxyalkylcellulose; |
| (c) | 5-7% | One or more cyclodextrins; |
| (d) | 0.1-5% | One or more pH adjusters; |
| (e) | 2-4% | One or more buffers; and |
| (f) | 0.1-3% | One or more stabilizers. |

The composition can comprise:

| | | |
|---|---|---|
| (a) | 1-5% | An NSAID; |
| (b) | 0.5-8% | A combination of a cellulose and an alkali metal |
| | | carboxyalkylcellulose; |
| (c) | 1-7% | One or more cyclodextrins; |
| (d) | 0.1-1% | One or more pH adjusters; |
| (e) | 1-15% | One or more buffers; and |
| (f) | 0.1-1% | One or more stabilizers. |

The composition can comprise:

| | | |
|---|---|---|
| (a) | 1-5% | An NSAID; |
| (b) | 0.5-8% | A combination of a cellulose and an alkali metal |
| | | carboxyalkylcellulose; |
| (c) | 1-7% | One or more cyclodextrins; |
| (d) | 0.1-1% | One or more pH adjusters; |
| (e) | 1-5% | One or more buffers; and |
| (f) | 0.1-1% | One or more stabilizers. |

The composition can comprise:

| | | |
|---|---|---|
| (a) | 1-5% | An NSAID; |
| (b) | 1-5% | A combination of a cellulose and an alkali metal |
| | | carboxyalkylcellulose; |
| (c) | 1-7% | One or more cyclodextrins; |
| (d) | 0.1-1% | One or more pH adjusters; |
| (e) | 1-5% | One or more buffers; and |
| (f) | 0.1-1% | One or more stabilizers. |

The composition can comprise:

| | |
|---|---|
| (a) 1-5% | An NSAID; |
| (b) 1-5% | A combination of a cellulose and an |
| | alkali metal carboxyalkylcellulose; |
| (c) 1-15% | One or more cyclodextrins; |
| (d) 0.1-10% | One or more pH adjusters; |
| (e) 0.1-15% | One or more buffers; |
| (f) 0.05-5% | One or more stabilisers; and |
| (g) 0.1-1% | Preservatives. |

The composition can comprise:

| | |
|---|---|
| (a) 1-5% | An NSAID; |
| (b) 0.5-8% | A combination of a cellulose and an |
| | alkali metal carboxyalkylcellulose; |
| (c) 1-7% | One or more cyclodextrins; |
| (d) 0.1-1% | One or more pH adjusters; |
| (e) 1-5% | One or more buffers; |
| (f) 0.1-1% | One or more stabilisers; and |
| (g) 0.1-1% | Preservatives. |

The composition can comprise:

| | |
|---|---|
| (a) 1-5% | An NSAID; |
| (b) 1-5% | A combination of a cellulose and an |
| | alkali metal carboxyalkylcellulose; |
| (c) 1-7% | One or more cyclodextrins; |
| (d) 0.1-1% | One or more pH adjusters; |
| (e) 1-5% | One or more buffers; |
| (f) 0.1-1% | One or more stabilisers; and |
| (g) 0.1-1% | Preservatives. |

The composition can comprise:

| | |
|---|---|
| (a) 1-5% | An NSAID; |
| (b) 1-5% | A combination of a cellulose and an |
| | alkali metal carboxyalkylcellulose; |
| (c) 1-15% | One or more cyclodextrins; |
| (d) 1-10% | One or more pH adjusters; |
| (e) 0.1-15% | One or more buffers; |
| (f) 0.05-5% | One or more stabilisers; and |
| (g) 0.1-1% | Preservatives. |

The composition can comprise:

| | |
|---|---|
| (a) 1-5% | Flurbiprofen; |
| (b) 1-5% | A combination of microcrystalline cellulose |
| | and sodium carboxymethylcellulose; |
| (c) 1-15% | Combination of beta-cyclodextrin and |
| | hydroxypropyl beta cyclodextrin; |
| (d) 0.1-5% | Sodium hydroxide; |
| (e) 1-10% | Disodium hydrogen phosphate; |
| (f) 0.1-5% | Citric acid monohydrate; and |
| (g) 0.05-0.5% | Xanthan gum. |

The composition can comprise:

| | |
|---|---|
| (a) 1-5% | Flurbiprofen; |
| (b) 0.5-8% | A combination of microcrystalline cellulose |
| | and sodium carboxymethylcellulose; |
| (c) 1-7% | Combination of beta-cyclodextrin and |
| | hydroxypropyl beta cyclodextrin; |
| (d) 0.1-1% | Sodium hydroxide; |
| (e) 1-5% | Disodium hydrogen phosphate; |
| (f) 0.1-1% | Citric acid monohydrate; and |
| (g) 0.05-0.5% | Xanthan gum. |

The composition can comprise:

| | |
|---|---|
| (a) 1-5% | Flurbiprofen; |
| (b) 1-5% | A combination of microcrystalline cellulose |
| | and sodium carboxymethylcellulose; |
| (c) 1-7% | Combination of beta-cyclodextrin and |
| | hydroxypropyl beta cyclodextrin; |
| (d) 0.1-1% | Sodium hydroxide; |
| (e) 1-5% | Disodium hydrogen phosphate; |
| (f) 0.1-1% | Citric acid monohydrate; and |
| (g) 0.05-0.5% | Xanthan gum. |

The composition can comprise:

| | |
|---|---|
| (a) 1-5% | Flurbiprofen; |
| (b) 1-5% | A combination of microcrystalline cellulose |
| | and sodium carboxymethylcellulose; |
| (c) 1-15% | Combination of beta-cyclodextrin and |
| | hydroxypropyl beta cyclodextrin; |
| (d) 0.1-5% | Sodium hydroxide; |
| (e) 1-10% | Disodium hydrogen phosphate; |
| (f) 0.1-5% | Citric acid monohydrate; |
| (g) 0.05-5% | Xanthan gum; |
| (h) 0.1-5% | Methyl paraben; and |
| (i) 0.1-5% | Propyl paraben. |

The composition can comprise:

| | |
|---|---|
| (a) 1-5% | Flurbiprofen; |
| (b) 1-5% | A combination of microcrystalline cellulose |
| | and sodium carboxymethylcellulose; |
| (c) 1-15% | Combination of beta-cyclodextrin and |
| | hydroxypropyl beta cyclodextrin; |
| (d) 0.1-5% | Sodium hydroxide; |
| (e) 1-10% | Disodium hydrogen phosphate; |
| (f) 0.1-5% | Citric acid monohydrate; |
| (g) 0.05-0.5% | Xanthan gum; |
| (h) 0.1-5% | Methyl paraben; and |
| (i) 0.1-5% | Propyl paraben. |

The composition can consist essentially of:

| | |
|---|---|
| (a) 1-5% | An NSAID; |
| (b) 1-5% | A combination of a cellulose and an |
| | alkali metal carboxyalkylcellulose; |
| (c) 1-15% | One or more cyclodextrins; |
| (d) 0.1-10% | One or more pH adjusters; and |
| (e) 0.1-15% | One or more buffers. |

The composition can consist essentially of:

| | |
|---|---|
| (a) 1-5% | An NSAID; |
| (b) 1-5% | A combination of a cellulose and an |
| | alkali metal carboxyalkylcellulose; |
| (c) 1-15% | One or more cyclodextrins; |
| (d) 1-10% | One or more pH adjusters; and |
| (e) 0.1-15% | One or more buffers. |

The composition can consist essentially of:

| | |
|---|---|
| (a) 1-5% | An NSAID; |
| (b) 0.5-8% | A combination of a cellulose and an |
| | alkali metal carboxyalkylcellulose; |
| (c) 1-10% | One or more cyclodextrins; |
| (d) 0.1-1% | One or more pH adjusters; and |
| (e) 1-5% | One or more buffers. |

The composition can consist essentially of:

| | |
|---|---|
| (a) 1-5% | An NSAID; |
| (b) 0.5-8% | A combination of a cellulose and an |
| | alkali metal carboxyalkylcellulose; |
| (c) 1-7% | One or more cyclodextrins; |
| (d) 0.1-1% | One or more pH adjusters; and |
| (e) 1-5% | One or more buffers. |

The composition can consist essentially of:

| | |
|---|---|
| (a) 1-5% | An NSAID; |
| (b) 1-5% | A combination of a cellulose and an |
| | alkali metal carboxyalkylcellulose; |
| (c) 1-7% | One or more cyclodextrins; |
| (d) 0.1-1% | One or more pH adjusters; and |
| (e) 1-5% | One or more buffers. |

The composition can consist essentially of:

| | |
|---|---|
| (a) 1-5% | An NSAID; |
| (b) 1-5% | A combination of a cellulose and an |
| | alkali metal carboxyalkylcellulose; |
| (c) 1-15% | One or more cyclodextrins; |
| (d) 0.1-10% | One or more pH adjusters; |
| (e) 0.1-15% | One or more buffers ; and |
| (f) 0.05-5% | One or more stabilisers. |

The composition can consist essentially of:

| | |
|---|---|
| (a) 1-5% | An NSAID; |
| (b) 1-5% | A combination of a cellulose and an alkali metal |
| | carboxyalkylcellulose; |
| (c) 1-15% | One or more cyclodextrins; |
| (d) 1-10% | One or more pH adjusters; |
| (e) 0.1-15% | One or more buffers; and |
| (f) 0.05-5% | One or more stabilisers. |

The composition can consist essentially of:

| | |
|---|---|
| (a) 1-5% | An NSAID; |
| (b) 0.5-8% | A combination of a cellulose and an alkali metal |
| | carboxyalkylcellulose; |
| (c) 1-10% | One or more cyclodextrins; |
| (d) 0.1-1% | One or more pH adjusters; |
| (e) 1-5% | One or more buffers; and |
| (f) 0.1-1% | One or more stabilisers. |

The composition can consist essentially of:

| | |
|---|---|
| (a) 1-5% | An NSAID; |
| (b) 0.5-8% | A combination of a cellulose and an alkali metal |
| | carboxyalkylcellulose; |
| (c) 1-7% | One or more cyclodextrins; |
| (d) 0.1-1% | One or more pH adjusters; |
| (e) 1-5% | One or more buffers; and |
| (f) 0.1-1% | One or more stabilisers. |

The composition can consist essentially of:

| | |
|---|---|
| (a) 1-5% | An NSAID; |
| (b) 1-5% | A combination of a cellulose and an alkali metal |
| | carboxyalkylcellulose; |
| (c) 1-7% | One or more cyclodextrins; |
| (d) 0.1-1% | One or more pH adjusters; |
| (e) 1-5% | One or more buffers; and |
| (f) 0.1-1% | One or more stabilisers. |

The composition can consist essentially of:

| | |
|---|---|
| (a) 1-5% | Flurbiprofen; |
| (b) 1-5% | A combination of a cellulose and an alkali metal |
| | carboxyalkylcellulose; |
| (c) 1-15% | One or more cyclodextrins; |
| (d) 0.1-15% | One or more buffers; and |
| (e) 0.05-5% | One or more stabilisers. |

The composition can consist essentially of:

| | |
|---|---|
| (a) 1-5% | Flurbiprofen; |
| (b) 1-5% | A combination of a cellulose and an alkali metal |
| | carboxyalkylcellulose; |
| (c) 1-15% | One or more cyclodextrins; |
| (d) 0.1-10% | One or more pH adjusters; and |
| (e) 0.1-15% | One or more buffers. |

The composition can consist essentially of:

| | |
|---|---|
| (a) 1-5% | Flurbiprofen; |
| (b) 0.5-8% | A combination of a cellulose and an alkali metal |
| | carboxyalkylcellulose; |
| (c) 1-7% | One or more cyclodextrins; |
| (d) 1-5% | One or more buffers; and |
| (e) 0.1-1% | One or more stabilisers. |

The composition can consist essentially of:

| | |
|---|---|
| (a) 1-5% | Flurbiproten; |
| (b) 1-5% | A combination of a cellulose and an alkali metal |
| | carboxyalkylcellulose; |
| (c) 1-7% | One or more cyclodextrins; |
| (d) 1-5% | One or more buffers; and |
| (e) 0.1-1% | One or more stabilisers. |

The composition can consist essentially of:

| | |
|---|---|
| (a) 1-5% | Flurbiprofen; |
| (b) 1-5% | A combination of a cellulose and an alkali metal |
| | carboxyalkylcellulose; |
| (c) 1-15% | One or more cyclodextrins; |
| (d) 0.1-10% | One or more pH adjusters; |
| (e) 0.1-15% | One or more buffers; and |
| (f) 0.05-5% | One or more stabilisers. |

The composition can consist essentially of:

| | |
|---|---|
| (a) 1-5% | Flurbiprofen; |
| (b) 0.5-8% | A combination of a cellulose and an alkali metal |
| | carboxyalkylcellulose; |
| (c) 1-10% | One or more cyclodextrins; |
| (d) 0.1-1% | One or more pH adjusters; |
| (e) 1-5% | One or more buffers; and |
| (f) 0.1-1% | One or more stabilisers. |

The composition can consist essentially of:

| | |
|---|---|
| (a) 1-5% | Flurbiprofen; |
| (b) 0.5-8% | A combination of a cellulose and an alkali metal |
| | carboxyalkylcellulose; |
| (c) 1-7% | One or more cyclodextrins; |
| (d) 0.1-1% | One or more pH adjusters; |
| (e) 1-5% | One or more buffers; and |
| (f) 0.1-1% | One or more stabilisers. |

The composition can consist essentially of:

| | |
|---|---|
| (a) 1-5% | Flurbiprofen; |
| (b) 1-5% | A combination of a cellulose and an alkali metal |
| | carboxyalkylcellulose; |
| (c) 1-7% | One or more cyclodextrins; |
| (d) 0.1-1% | One or more pH adjusters; |
| (e) 1-5% | One or more buffers; and |
| (f) 0.1-1% | One or more stabilisers. |

The composition can consist essentially of:

| | |
|---|---|
| (a) 1-5% | Flurbiprofen; |
| (b) 1-5% | A combination of a cellulose and an alkali metal |
| | carboxyalkylcellulose; |
| (c) 1-5% | One or more cyclodextrins; |
| (d) 1-10% | One or more pH adjusters; |
| (e) 0.1-15% | One or more buffers; and |
| (f) 0.05-5% | One or more stabilisers. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-15% | A combination of beta-cyclodextrin and hydroxypropyl beta |
| | cyclodextrin; |
| (d) 0.1-5% | Sodium hydroxide; |
| (e) 1-10% | Disodium hydrogen phosphate; and |
| (f) 0.1-5% | Citric acid monohydrate. |

The composition can consist essentially of:

| | |
|---|---|
| (a) 1-2% | Flurbiprofen; |
| (b) 2-4% | A combination of a cellulose and an alkali metal |
| | carboxyalkylcellulose; |
| (c) 5-7% | One or more cyclodextrins; |
| (d) 0.1-0.5% | One or more pH adjusters; and |
| (e) 2-4% | One or more buffers. |

The composition can consist essentially of:

| | |
|---|---|
| (a) 1-2% | Flurbiprofen; |
| (b) 2-4% | A combination of a cellulose and an alkali metal |
| | carboxyalkylcellulose; |
| (c) 5-7% | One or more cyclodextrins; |
| (d) 0.1-5% | One or more pH adjusters; |

The composition can consist essentially of:

| | |
|---|---|
| (a) 1-5% | Flurbiprofen; |
| (b) 0.5-8% | A combination of microcrystalline cellulose and sodium |
| | carboxymethylcellulose; |
| (c) 1-10% | A combination of beta-cyclodextrin and hydroxypropyl |
| | beta cyclodextrin; |
| (d) 0.1-1% | Sodium hydroxide; |
| (e) 1-5% | Disodium hydrogen phosphate; and |
| (f) 0.1-1% | Citric acid monohydrate. |

The composition can consist essentially of:

| | |
|---|---|
| (a) 1-5% | Flurbiprofen; |
| (b) 0.5-8% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; and |
| (f) 0.1-1% | Citric acid monohydrate. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; and |
| (f) 0.1-1% | Citric acid monohydrate. |

The composition can consist essentially of:

| | |
|---|---|
| (a) 1-2% | Flurbiprofen; |
| (b) 2-4% | A combination of microcrystalline cellulose and sodium |
| | carboxymethylcellulose; |
| (c) 5-7% | A combination of beta-cyclodextrin and hydroxypropyl |
| | beta cyclodextrin; |
| (d) 0.1-5% | Sodium hydroxide; |
| (e) 1-10% | Disodium hydrogen phosphate; and |
| (f) 0.1-5% | Citric acid monohydrate. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-15% | A combination of beta-cyclodextrin and hydroxypropyl |
| | beta cyclodextrin; |
| (d) 0.1-5% | Sodium hydroxide; |
| (e) 1-10% | Disodium hydrogen phosphate; |
| (f) 0.1-5% | Citric acid monohydrate; and |
| (g) 0.05-5% | Xanthan gum. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-15% | A combination of beta-cyclodextrin and hydroxypropyl |
| | beta cyclodextrin; |
| (d) 0.1-5% | Sodium hydroxide; |
| (e) 1-10% | Disodium hydrogen phosphate; |
| (f) 0.1-5% | Citric acid monohydrate; and |
| (g) 0.05-0.5% | Xanthan gum. |

The composition can consist essentially of:

| | |
|---|---|
| (a) 1-5% | Flurbiprofen; |
| (b) 0.5-8% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; |
| (f) 0.1-1% | Citric acid monohydrate; and |
| (g) 0.1-0.5% | Xanthan gum. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; |
| (f) 0.1-1% | Citric acid monohydrate; and |
| (g) 0.1-0.5% | Xanthan gum. |

The composition can consist essentially of:

| | |
|---|---|
| (a) 1-2% | Flurbiprofen; |
| (b) 2-4% | A combination of microcrystalline cellulose and sodium |
| | carboxymethylcellulose; |
| (c) 5-7% | A combination of beta-cyclodextrin and hydroxypropyl |
| | beta cyclodextrin; |
| (d) 0.1-5% | Sodium hydroxide; |
| (e) 1-10% | Disodium hydrogen phosphate; |
| (f) 0.1-5% | Citric acid monohydrate; and |
| (g) 0.1-0.3% | Xanthan gum. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-15% | A combination of beta-cyclodextrin and hydroxypropyl |
| | beta cyclodextrin; |
| (d) 0.1-5% | Sodium hydroxide; |
| (e) 1-10% | Disodium hydrogen phosphate; |
| (f) 0.1-5% | Citric acid monohydrate; |
| (g) 0.1-5% | Methyl paraben; and |
| (h) 0.01-5% | Propyl paraben. |

The composition can consist essentially of:

| | |
|---|---|
| (a) 1-5% | Flurbiprofen; |
| (b) 0.5-8% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; |
| (f) 0.1-1% | Citric acid monohydrate; |
| (g) 0.1-5% | Methyl paraben; and |
| (h) 0.01-5% | Propyl paraben. |

The composition can consist essentially of:

| | |
|---|---|
| (a) 1-5% | Flurbiprofen; |
| (b) 0.5-8% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; |
| (f) 0.1-1% | Citric acid monohydrate; |
| (g) 0.1-0.5% | Xanthan gum; |
| (h) 0.1-5% | Methyl paraben; and |
| (i) 0.01-5% | Propyl paraben. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; |
| (f) 0.1-1% | Citric acid monohydrate; |
| (g) 0.1-5% | Methyl paraben; and |
| (h) 0.01-5% | Propyl paraben. |

The composition can consist essentially of:

| | |
|---|---|
| (a) 1-5% | Flurbiprofen; |
| (b) 1-5% | A combination of microcrystalline cellulose and sodium |
| | carboxymethylcellulose; |
| (c) 1-7% | A combination of beta-cyclodextrin and hydroxypropyl |
| | beta cyclodextrin; |
| (d) 0.1-1% | Sodium hydroxide; |
| (e) 1-5% | Disodium hydrogen phosphate; |
| (f) 0.1-1% | Citric acid monohydrate; |
| (g) 0.1-0.5% | Xanthan gum; |
| (h) 0.1-5% | Methyl paraben; and |
| (i) 0.01-5% | Propyl paraben. |

According to an aspect of the present invention there is provided a sprayable aqueous thixotropic pharmaceutical composition in the form of a solution wherein the active pharmaceutical ingredient includes a charged functional group, a thixotropic agent and a solubilising agent.

The pharmaceutically active ingredient can be selected from NSAIDs or pharmaceutically acceptable salts thereof. A preferred NSAID is taken from the group consisting of flurbiprofen, ketoprofen or diclofenac. A more preferred NSAID is flurbiprofen.

The composition can comprise up to about 5% NSAID by weight of the composition. Typically, the amount of NSAID is from 0.5% by weight of the composition to 3% by weight of the composition. More typically, the amount of NSAID is from 1% by weight of the composition to 2% by weight of the composition.

The composition can comprise 10-25 mg/ml of NSAID. Preferably the composition comprises 15-18 mg/ml of NSAID. More preferably the composition comprises 16-17 mg/ml of NSAID.

The thixotropic agent can be present in an amount of 0.1-15% by weight of the composition. Preferably, the thixotropic agent can be present in an amount of 0.5-10% by weight of the composition. More preferably, the thixotropic agent can be present in an amount of 0.5-8% by weight of the composition. More preferably, Preferably, the thixotropic agent can be present in an amount of 1-5% by weight of the composition.

The thixotropic agent can be selected from combinations of one or more celluloses and one or more alkali metal carboxyalkylcelluloses, carrageenan and xanthan gum. Preferably, the thixotropic agent is selected from a combination of one or more celluloses and one or more alkali metal carboxyalkylcelluloses.

A preferred combination of cellulose and alkali metal carboxyalkylcellulose is microcrystalline cellulose and sodium carboxymethylcellulose. Typically this combination contains 85-95 weight percent microcrystalline cellulose and 5-15 weight percent sodium carboxymethylcellulose. More typically this combination contains 86-92 weight percent microcrystalline cellulose and 8-14 weight percent sodium carboxymethylcellulose.

Typically, the compositions of the present invention contain at least about 1.5 weight percent of the cellulose/carboxyalkylcellulose combination up to about 10 weight percent to avoid producing high viscosities which impede spraying with the usual devices. Preferably, the compositions contain about 1.5 to about 5 weight percent of the mixture will be included. More preferably, the amount will be about 2.0 to about 3.0 weight percent. Most preferably, the composition contains about 2.2 to about 2.8 weight percent.

The solubilising agent can be present in the composition in an amount of 0.1-15 w/w %. The solubilising agent can be present in the composition in an amount of 1-10 w/w %. Typically, the solubilising agent can be present in the composition in an amount of 3-7 w/w %. Typically, the solubilising agent can be present in an amount of 5-7 w/w %. More typically, the solubilising agent can be present in an amount of 6 w/w %.

The solubilising agent is typically selected from cyclodextrins, or combinations thereof. Other solubilising agents include non-ionic surfactants, such as Tween, and propylene glycol.

The cyclodextrin can be selected from α, β, γ cyclodextrin and derivatives thereof. Cyclodextrins for use in the present invention include the natural cyclodextrins and their derivatives, including the alkylated and hydroxyalkylated derivatives and the branched cyclodextrins. derivatives bearing sugar residues are of special interest. Especially useful herein are the hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α, β, γ-cyclodextrin. Specific cyclodextrin derivatives for use herein include α-cyclodextrin, β-cyclodextrin, γ-cyclodextrin, methyl-α-cyclodextrin, hydroxyethyl-α-cyclodextrin, hydroxypropyl-α-cyclodextrin, dihydroxypropyl-α-cyclodextrin, methyl-β-cyclodextrin, hydroxyethyl-β-cyclodextrin, hydroxypropyl-β-cyclodextrin, dihydroxypropyl-β-cyclodextrin, methyl-γ-cyclodextrin, hydroxyethyl-γ-cyclodextrin, hydroxypropyl-γ-cyclodextrin and dihydroxypropyl-γ-cyclodextrin.

Preferably, the cyclodextrin comprises more than one cyclodextrin. More preferably the cyclodextrin comprises 2 cyclodextrins.

Typically, the molar ratio of cyclodextrins is from 1:1 to 1:2 by weight percent of the composition. Preferably, the molar ratio of cyclodextrins is 1:1.9.

Alternatively the molar ratio of the cyclodextrins is 1:2.

The cyclodextrin can comprise a combination of β-cyclodextrin and hydroxypropyl-β-cyclodextrin.

The composition can comprise one or more stabilisers. The one or more stabilisers can be selected from ethyl cellulose, hydroxypropyl methyl cellulose and xanthan gum. A preferred stabiliser is xanthan gum.

The one or more stabilisers can be present at an amount of 0.05-5% w/w. Preferably the amount of the one or more stabilisers is 0.1-1% w/w. More preferably the amount of the stabilisers is 0.15-0.5% w/w.

The pH of the composition is preferably from 6.0-8.0. More preferably from 7.0-7.6. Most preferably the pH is about 7.4.

The composition can further include one or more buffers. The one or more buffers is selected to ensure that the pH of the composition is between about 6.0 and 8.0. Preferably the one or more buffers is selected to ensure that the pH of the composition is between 7.0 and 7.6. More preferably the one or more buffers is selected to ensure that the pH of the composition is about 7.4.

Preferably, the one or more buffers is selected from citric acid, disodium hydrogen phosphate, sodium hydrogen phosphate and combinations thereof.

Typically, the one or more buffers comprises citric acid and disodium hydrogen phosphate. More typically, the composition comprises a combination of citric acid and disodium hydrogen phosphate.

Preferably, the composition comprises from 0.1-5% w/w of citric acid and 1-10% w/w disodium hydrogen phosphate. More preferably, the composition comprises from 0.1-0.5% w/w of citric acid and 1-5% w/w disodium hydrogen phosphate.

The composition can further include additional pharmaceutically acceptable excipients selected from sweeteners, aesthetic agents, flavours, preservatives, pH adjusters and non-ionic buffers.

According to an aspect of the present invention there is provided a thixotropic composition comprising an active pharmaceutical ingredient having a low shear viscosity of 30-80 Pascal seconds (Pa·s) and high shear viscosity of 0.01-5 Pascals seconds (Pa·s).

Preferably the low shear viscosity of the composition is between 40 Pascal seconds (Pa·s) and 70 Pascal seconds (Pa·s). More preferably the low shear viscosity of the composition is between 45 Pascal seconds (Pa·s) and 65 Pascal seconds (Pa·s). Most preferably the low shear viscosity of the composition is between 50 Pascal seconds (Pa·s) and 60 Pascal seconds (Pa·s).

Preferably the high shear viscosity of the composition is between 0.05 Pascal seconds (Pa·s) and 2.5 Pascal seconds (Pa·s). More preferably the high shear viscosity of the composition is between 0.075 Pascal seconds (Pa·s) and 1.5 Pascal seconds (Pa·s). Most preferably the high shear viscosity of the composition is between 0.1 Pascal seconds (Pa·s)s and 1 Pascal seconds (Pa·s).

Preferably the composition has a high shear viscosity between 0.1 Pascal seconds (Pa·s) and 1 Pascal seconds (Pa·s) and a low shear viscosity between 50 Pascal seconds (Pa·s) and 60 Pascal seconds (Pa·s).

The pharmaceutically active ingredient can be selected from NSAIDs or pharmaceutically acceptable salts thereof. A preferred NSAID is taken from the group consisting of flurbiprofen, ketoprofen or diclofenac. A more preferred NSAID is flurbiprofen.

Other pharmaceutically active ingredients that can be used are hexylresorcinol, benzocaine, dextromethorphan, menthol lidocaine, amyl metacresol and 2,4-dichlorobenzyl alcohol or pharmaceutically acceptable salts thereof.

The composition can comprise up to about 5% NSAID by weight of the composition. Typically, the amount of NSAID is from 0.5% by weight of the composition to 3% by weight of the composition. More typically, the amount of NSAID is from 1% by weight of the composition to 2% by weight of the composition.

The composition can comprise 10-25 mg/ml of NSAID. Preferably the composition comprises 15-18 mg/ml of NSAID. More preferably the composition comprises 16-17 mg/ml of NSAID.

When the active is selected to be hexylrescinol the composition can comprise 2-5 mg/ml of the active. When the active is selected to be benzocaine the composition can comprise 10-20 mg/ml. When the active is selected to be dextromethorphan the composition can comprise 15-30 mg/ml. When active is selected to be menthol the composition can comprise 5-15 mg/ml. When the active is selected to be lidocaine the composition can comprise 2-5 mg/ml. When the active is selected to be amyl metacresol the composition can comprise 0.5-1.5 mg/ml. When the active is selected to be 2,4-dichlorobenzyl alcohol the composition can comprise 1-2.5 mg/ml.

Preferably, the composition has a very rapid rate of viscosity recovery, following withdrawal of the shearing force. A ‘very rapid rate of viscosity recovery’ means that the viscosity of the composition returns to about 90% of its initial viscosity within 30 s following withdrawal of the shearing force. More preferably, the viscosity of the composition returns to about 90% of its initial viscosity within 10 s following withdrawal of the shear force. Most preferably, the viscosity of the composition returns to about 90% of its initial viscosity within 5 s following withdrawal of the shear force. As used herein the term ‘initial viscosity’ is the viscosity of the composition prior to the application of a shear force.

The thixotropic agent can be present in an amount of 0.1-15% by weight of the composition. Preferably, the thixotropic agent can be present in an amount of 0.5-10% by weight of the composition. More preferably, the thixotropic agent can be present in an amount of 0.5-8% by weight of the composition. More preferably, Preferably, the thixotropic agent can be present in an amount of 1-5% by weight of the composition.

The thixotropic agent can be selected from combinations of one or more celluloses and one or more alkali metal carboxyalkylcelluloses, carrageenan and xanthan gum. Preferably, the thixotropic agent is selected from a combination of one or more celluloses and one or more alkali metal carboxyalkylcelluloses.

A preferred combination of cellulose and alkali metal carboxyalkylcellulose is microcrystalline cellulose and sodium carboxymethylcellulose. Typically this combination contains 85-95 weight percent microcrystalline cellulose and 5-15 weight percent sodium carboxymethylcellulose. More typically this combination contains 86-92 weight percent microcrystalline cellulose and 8-14 weight percent sodium carboxymethylcellulose.

Typically, the compositions of the present invention contain at least about 1.5 weight percent of the cellulose/carboxyalkylcellulose combination up to about 10 weight percent to avoid producing high viscosities which impede spraying with the usual devices. Preferably, the compositions contain about 1.5 to about 5 weight percent of the mixture will be included. More preferably, the amount will be about 2.0 to about 3.0 weight percent. Most preferably, the composition contains about 2.2 to about 2.8 weight percent.

The solubilising agent can be present in the composition in an amount of 0.1-15 w/w %. The solubilising agent can be present in the composition in an amount of 1-10 w/w %. Typically, the solubilising agent can be present in the composition in an amount of 3-7 w/w %. Typically, the solubilising agent can be present in an amount of 5-7 w/w %. More typically, the solubilising agent can be present in an amount of 6 w/w %.

The solubilising agent is typically selected from cyclodextrins, or combinations thereof. Other solubilising agents include non-ionic surfactants, such as Tween, and propylene glycol.

The cyclodextrin can be selected from α, β, γ cyclodextrin and derivatives thereof. Cyclodextrins for use in the present invention include the natural cyclodextrins and their derivatives, including the alkylated and hydroxyalkylated derivatives and the branched cyclodextrins. derivatives bearing sugar residues are of special interest. Especially useful herein are the hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α, β, γ-cyclodextrin. Specific cyclodextrin derivatives for use herein include α-cyclodextrin, β-cyclodextrin, γ-cyclodextrin, methyl-α-cyclodextrin, hydroxyethyl-α-cyclodextrin, hydroxypropyl-α-cyclodextrin, dihydroxypropyl-α-cyclodextrin, methyl-β-cyclodextrin, hydroxyethyl-β-cyclodextrin, hydroxypropyl-β-cyclodextrin, dihydroxypropyl-β-cyclodextrin, methyl-γ-cyclodextrin, hydroxyethyl-γ-cyclodextrin, hydroxypropyl-γ-cyclodextrin and dihydroxypropyl-γ-cyclodextrin.

Preferably, the cyclodextrin comprises more than one cyclodextrin. More preferably, the cyclodextrin comprises 2 cyclodextrins.

Typically, the molar ratio of cyclodextrins is from 1:1 to 1:2 by weight percent of the composition. Preferably, the molar ratio of cyclodextrins is 1:1.9.

Alternatively the molar ratio of the cyclodextrins is 1:2.

The cyclodextrin can comprise a combination of β-cyclodextrin and hydroxypropyl-β-cyclodextrin.

The composition can comprise one or more stabilisers. The one or more stabilisers can be selected from ethyl cellulose, hydroxypropyl methyl cellulose and xanthan gum. A preferred stabiliser is xanthan gum.

The one or more stabilisers can be present at an amount of 0.05-5% w/w. Preferably the amount of the one or more stabilisers is 0.1-1% w/w. More preferably the amount of the stabilisers is 0.15-0.5% w/w.

The pH of the composition is preferably from 6.0-8.0. More preferably from 7.0-7.6. Most preferably the pH is about 7.4.

The composition can further include one or more buffers. The one or more buffers is selected to ensure that the pH of the composition is between about 6.0 and 8.0. Preferably the one or more buffers is selected to ensure that the pH of the composition is between 7.0 and 7.6. More preferably the one or more buffers is selected to ensure that the pH of the composition is about 7.4.

The one or more buffers can be present at an amount of 0.1-15% w/w. Preferably the amount of the one or more buffers is 1-5% w/w. More preferably the amount of the buffers is 2-4% w/w.

Preferably, the one or more buffers is selected from citric acid, disodium hydrogen phosphate, sodium hydrogen phosphate and combinations thereof.

Typically, the one or more buffers comprises citric acid and disodium hydrogen phosphate. More typically, the composition comprises a combination of citric acid and disodium hydrogen phosphate.

Preferably, the composition comprises from 0.1-5% w/w of citric acid and 1-10% w/w disodium hydrogen phosphate. More preferably, the composition comprises from 0.1-0.5% w/w of citric acid and 1-5% w/w disodium hydrogen phosphate.

The composition can further include additional pharmaceutically acceptable excipients selected from sweeteners, aesthetic agents, flavours, preservatives, pH adjusters and non-ionic buffers.

The thixotropic composition can be in the form of a solution comprising 1-5% by weight of flurbiprofen or pharmaceutically acceptable salt thereof, 5-7% by weight of a solubilising agent selected from cyclodextrins or combinations thereof, 1.5-5% by weight of a thixotropic agent selected from combinations of one or more celluloses and one or more alkali metal carboxyalkylcelluloses, 0.1-1% by weight of one or more stabilisers selected from ethyl cellulose, hydroxypropyl methyl cellulose and xanthan gum, and 1-5% by weight of one or more buffers selected from citric acid, disodium hydrogen phosphate, sodium hydrogen phosphate and combinations thereof has wherein the composition has a low shear viscosity of 30-80 Pascal seconds (Pa·s) and high shear viscosity of 0.01-5 Pascals seconds (Pa·s). Preferably, the amount of flurbiprofen is from 1-2% by weight of the composition. Preferably the amount of thixotropic agent is 2-3% by weight of the composition. Preferably, the amount of stabiliser is 0.1-0.5% by weight of the composition. Preferably the amount of buffer is 2-4% by weight of the composition. Preferably the composition has a high shear viscosity between 0.1 Pascal seconds (Pa·s) and 1 Pascal seconds (Pa·s) and a low shear viscosity between 50 Pascal seconds (Pa·s) and 60 Pascal seconds (Pa·s).

The thixotropic composition can be in the form of a solution consisting essentially of 1-5% by weight of flurbiprofen or pharmaceutically acceptable salt thereof, 5-7% by weight of a solubilising agent selected from cyclodextrins or combinations thereof, 1.5-5% by weight of a thixotropic agent selected from combinations of one or more celluloses and one or more alkali metal carboxyalkylcelluloses, 0.1-1% by weight of one or more stabilisers selected from ethyl cellulose, hydroxypropyl methyl cellulose and xanthan gum, and 1-5% by weight of one or more buffers selected from citric acid, disodium hydrogen phosphate, sodium hydrogen phosphate and combinations thereof wherein the composition has a low shear viscosity of 30-80 Pascal seconds (Pa·s) and high shear viscosity of 0.01-5 Pascals seconds (Pa·s). Preferably, the amount of flurbiprofen is from 1-2% by weight of the composition. Preferably the amount of thixotropic agent is 2-3% by weight of the composition. Preferably, the amount of stabiliser is 0.1-0.5% by weight of the composition. Preferably the amount of buffer is 2-4% by weight of the composition. Preferably the composition has a high shear viscosity between 0.1 Pascal seconds (Pa·s) and 1 Pascal seconds (Pa·s) and a low shear viscosity between 50 Pascal seconds (Pa·s) and 60 Pascal seconds (Pa·s).

According to an aspect of the present invention there is provided a thixotropic composition which comprises an NSAID or pharmaceutically acceptable salt thereof wherein the composition has a yield point of 1-30 Pa.

Preferably the yield point is 2-8 Pa.

Alternatively, the yield point can be 10-16 Pa.

The pharmaceutically active ingredient can be selected from NSAIDs or pharmaceutically acceptable salts thereof. A preferred NSAID is taken from the group consisting of flurbiprofen, ketoprofen or diclofenac. A more preferred NSAID is flurbiprofen.

Other pharmaceutically active ingredients that can be used are hexylresorcinol, benzocaine, dextromethorphan, menthol lidocaine, amyl metacresol and 2,4-dichlorobenzyl alcohol or pharmaceutically acceptable salts thereof.

The composition can comprise up to about 5% NSAID by weight of the composition. Typically, the amount of NSAID is from 0.5% by weight of the composition to 3% by weight of the composition. More typically, the amount of NSAID is from 1% by weight of the composition to 2% by weight of the composition.

The composition can comprise 10-25 mg/ml of NSAID. Preferably the composition comprises 15-18 mg/ml of NSAID. More preferably the composition comprises 16-17 mg/ml of NSAID.

When the active is selected to be hexylrescinol the composition can comprise 2-5 mg/ml of the active. When the active is selected to be benzocaine the composition can comprise 10-20 mg/ml. When the active is selected to be dextromethorphan the composition can comprise 15-30 mg/ml. When active is selected to be menthol the composition can comprise 5-15 mg/ml. When the active is selected to be lidocaine the composition can comprise 2-5 mg/ml. When the active is selected to be amyl metacresol the composition can comprise 0.5-1.5 mg/ml. When the active is selected to be 2,4-dichlorobenzyl alcohol the composition can comprise 1-2.5 mg/ml.

Preferably, the composition has a very rapid rate of viscosity recovery, following withdrawal of the shearing force. A ‘very rapid rate of viscosity recovery’ means that the viscosity of the composition returns to about 90% of its initial viscosity within 30 s following withdrawal of the shearing force. More preferably, the viscosity of the composition returns to about 90% of its initial viscosity within 10 s following withdrawal of the shear force. Most preferably, the viscosity of the composition returns to about 90% of its initial viscosity within 5 s following withdrawal of the shear force. As used herein the term ‘initial viscosity’ is the viscosity of the composition prior to the application of a shear force.

The thixotropic agent can be present in an amount of 0.1-15% by weight of the composition. Preferably, the thixotropic agent can be present in an amount of 0.5-10% by weight of the composition. More preferably, the thixotropic agent can be present in an amount of 0.5-8% by weight of the composition. More preferably, Preferably, the thixotropic agent can be present in an amount of 1-5% by weight of the composition.

The thixotropic agent can be selected from combinations of one or more celluloses and one or more alkali metal carboxyalkylcelluloses, carrageenan and xanthan gum. Preferably, the thixotropic agent is selected from a combination of one or more celluloses and one or more alkali metal carboxyalkylcelluloses.

A preferred combination of cellulose and alkali metal carboxyalkylcellulose is microcrystalline cellulose and sodium carboxymethylcellulose. Typically this combination contains 85-95 weight percent microcrystalline cellulose and 5-15 weight percent sodium carboxymethylcellulose. More typically this combination contains 86-92 weight percent microcrystalline cellulose and 8-14 weight percent sodium carboxymethylcellulose.

Typically, the compositions of the present invention contain at least about 1.5 weight percent of the cellulose/carboxyalkylcellulose combination up to about 10 weight percent to avoid producing high viscosities which impede spraying with the usual devices. Preferably, the compositions contain about 1.5 to about 5 weight percent of the mixture will be included. More preferably, the amount will be about 2.0 to about 3.0 weight percent. Most preferably, the composition contains about 2.2 to about 2.8 weight percent.

Typically the yield point is 2-8 when the composition comprises from 1.5 to 2.4% w/w of a combination of a cellulose and an alkali metal carboxyalkylcellulose.

Typically the yield point is 10-20 when the composition comprises from 2.4 to 3.0% w/w of a combination of a cellulose and an alkali metal carboxyalkylcellulose. More typically the yield point is 10-16 when the composition comprises from 2.4 to 3.0% w/w of a combination of a cellulose and an alkali metal carboxyalkylcellulose.

The solubilising agent can be present in the composition in an amount of 0.1-15 w/w %. The solubilising agent can be present in the composition in an amount of 1-10 w/w %. Typically, the solubilising agent can be present in the composition in an amount of 3-7 w/w %. Typically, the solubilising agent can be present in an amount of 5-7 w/w %. More typically, the solubilising agent can be present in an amount of 6 w/w %.

The solubilising agent is typically selected from cyclodextrins, or combinations thereof. Other solubilising agents include non-ionic surfactants, such as Tween, and propylene glycol.

The cyclodextrin can be selected from α, β, γ cyclodextrin and derivatives thereof. Cyclodextrins for use in the present invention include the natural cyclodextrins and their derivatives, including the alkylated and hydroxyalkylated derivatives and the branched cyclodextrins. derivatives bearing sugar residues are of special interest. Especially useful herein are the hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α, β, γ-cyclodextrin. Specific cyclodextrin derivatives for use herein include α-cyclodextrin, β-cyclodextrin, γ-cyclodextrin, methyl-α-cyclodextrin, hydroxyethyl-α-cyclodextrin, hydroxypropyl-α-cyclodextrin, dihydroxypropyl-α-cyclodextrin, methyl-β-cyclodextrin, hydroxyethyl-β-cyclodextrin, hydroxypropyl-β-cyclodextrin, dihydroxypropyl-β-cyclodextrin, methyl-γ-cyclodextrin, hydroxyethyl-γ-cyclodextrin, hydroxypropyl-γ-cyclodextrin and dihydroxypropyl-γ-cyclodextrin.

Preferably, the cyclodextrin comprises more than one cyclodextrin. More preferably, the cyclodextrin comprises 2 cyclodextrins.

Typically, the molar ratio of cyclodextrins is from 1:1 to 1:2 by weight percent of the composition. Preferably, the molar ratio of cyclodextrins is 1:1.9.

Alternatively the molar ratio of the cyclodextrins is 1:2.

The cyclodextrin can comprise a combination of β-cyclodextrin and hydroxypropyl-β-cyclodextrin.

The pH of the composition is preferably from 6.0-8.0. More preferably from 7.0-7.6. Most preferably the pH is about 7.4.

The composition can comprise one or more stabilisers. The one or more stabilisers can be selected from ethyl cellulose, hydroxypropyl methyl cellulose and xanthan gum. A preferred stabiliser is xanthan gum.

The one or more stabilisers can be present at an amount of 0.05-5% w/w. Preferably the amount of the one or more stabilisers is 0.1-1% w/w. More preferably the amount of the stabilisers is 0.15-0.5% w/w.

The pH of the composition is preferably from 6.0-8.0. More preferably from 7.0-7.6. Most preferably the pH is about 7.4.

The composition can further include one or more buffers. The one or more buffers is selected to ensure that the pH of the composition is between about 6.0 and 8.0. Preferably the one or more buffers is selected to ensure that the pH of the composition is between 7.0 and 7.6. More preferably the one or more buffers is selected to ensure that the pH of the composition is about 7.4.

The one or more buffers can be present at an amount of 0.1-15% w/w. Preferably the amount of the one or more buffers is 1-5% w/w. More preferably the amount of the buffers is 2-4% w/w.

Preferably, the one or more buffers is selected from citric acid, disodium hydrogen phosphate, sodium hydrogen phosphate and combinations thereof.

Typically, the one or more buffers comprises citric acid and disodium hydrogen phosphate. More typically, the composition comprises a combination of citric acid and disodium hydrogen phosphate.

Preferably, the composition comprises from 0.1-5% w/w of citric acid and 1-10% w/w disodium hydrogen phosphate. More preferably, the composition comprises from 0.1-0.5% w/w of citric acid and 1-5% w/w disodium hydrogen phosphate.

The composition can further include additional pharmaceutically acceptable excipients selected from sweeteners, aesthetic agents, flavours, preservatives, pH adjusters and non-ionic buffers.

The thixotropic composition can be in the form of a solution comprising 1-5% by weight of flurbiprofen or pharmaceutically acceptable salt thereof, 5-7% by weight of a solubilising agent selected from cyclodextrins or combinations thereof, 1.5-5% by weight of a thixotropic agent selected from combinations of one or more celluloses and one or more alkali metal carboxyalkylcelluloses, 0.1-1% by weight of one or more stabilisers selected from ethyl cellulose, hydroxypropyl methyl cellulose and xanthan gum, and 1-5% by weight of one or more and buffers selected from citric acid, disodium hydrogen phosphate, sodium hydrogen phosphate and combinations thereof wherein the composition has a yield point of 1-30 Pa. Preferably, the amount of flurbiprofen is from 1-2% by weight of the composition.

In a preferred embodiment the amount of thixotropic agent is 2.5-3% by weight of the composition, the amount of stabiliser is 0.1-0.5% by weight of the composition, the amount of buffer is 2-4% by weight of the composition and the composition has a yield point of 10-16 Pa.

In an alternative embodiment, the amount of flurbiprofen is from 1-2% by weight of the composition, the amount of thixotropic agent is 2-2.4% by weight of the composition, the amount of stabiliser is 0.1-1% by weight of the composition, the amount of buffer is 2-4% by weight of the composition and the composition has a yield point of 2-8 Pa.

The thixotropic composition can be in the form of a solution consisting essentially of 1-5% by weight of flurbiprofen or pharmaceutically acceptable salt thereof, 5-7% by weight of a solubilising agent selected from cyclodextrins or combinations thereof, 1.5-5% by weight of a thixotropic agent selected from combinations of one or more celluloses and one or more alkali metal carboxyalkylcelluloses, 0.1-1% by weight of one or more stabilisers selected from ethyl cellulose, hydroxypropyl methyl cellulose and xanthan gum, and 1-5% by weight of one or more and buffers selected from citric acid, disodium hydrogen phosphate, sodium hydrogen phosphate and combinations thereof wherein the composition has a yield point of 1-30 Pa. In a preferred embodiment the amount of thixotropic agent is 2.5-3% by weight of the composition, the amount of stabiliser is 0.1-1% by weight of the composition, the amount of buffer is 2-4% by weight of the composition and the composition has a yield point of 10-16 Pa. In an alternative embodiment, the amount of flurbiprofen is from 1-2% by weight of the composition, the amount of thixotropic agent is 2-2.4% by weight of the composition, the amount of stabiliser is 0.1-0.5% by weight of the composition, the amount of buffer is 2-4% by weight of the composition and the composition has a yield point of 2-8 Pa.

According to an aspect of the present invention there is provided the use of a composition comprising a pharmaceutically active ingredient as described in the previous aspects for the treatment of sore throat, including inflammation of the throat and pharyngeal irritations.

The pharmaceutically active ingredient can be selected from NSAIDs or pharmaceutically acceptable salts thereof. A preferred NSAID is taken from the group consisting of flurbiprofen, ketoprofen or diclofenac. A more preferred NSAID is flurbiprofen.

Other pharmaceutically active ingredients that can be used are hexylresorcinol, benzocaine, dextromethorphan, menthol lidocaine, amyl metacresol and 2,4-dichlorobenzyl alcohol or pharmaceutically acceptable salts thereof.

The composition can comprise up to about 5% NSAID by weight of the composition. Typically, the amount of NSAID is from 0.5% by weight of the composition to 3% by weight of the composition. More typically, the amount of NSAID is from 1% by weight of the composition to 2% by weight of the composition.

The NSAID is administered locally in a dose of 1-20 mg. Preferably the dose is 5-15 mg of NSAID. More preferably the dose is 8-10 mg of NSAID.

The composition comprises 10-25 mg/ml of NSAID. Preferably the composition comprises 15-18 mg/ml of NSAID. More preferably the composition comprises 16-17 mg/ml of NSAID.

When the active is selected to be hexylrescinol the composition can comprise 2-5 mg/ml of the active. When the active is selected to be benzocaine the composition can comprise 10-20 mg/ml. When the active is selected to be dextromethorphan the composition can comprise 15-30 mg/ml. When active is selected to be menthol the composition can comprise 5-15 mg/ml. When the active is selected to be lidocaine the composition can comprise 2-5 mg/ml. When the active is selected to be amyl metacresol the composition can comprise 0.5-1.5 mg/ml. When the active is selected to be 2,4-dichlorobenzyl alcohol the composition can comprise 1-2.5 mg/ml.

According to an aspect of the present invention there is provided a method of manufacturing a composition as described in the first aspect comprising the steps of:
(a) providing a first liquid composition comprising the pharmaceutically active agent;
(b) providing a second liquid composition comprising the thixotropic agent; and
(c) mixing the first composition with the second composition.

Typically, the first composition is added to the second composition.

The pharmaceutically active ingredient can be selected from NSAIDs or pharmaceutically acceptable salts thereof. A preferred NSAID is taken from the group consisting of flurbiprofen, ketoprofen or diclofenac. A more preferred NSAID is flurbiprofen.

Other pharmaceutically active ingredients that can be used are hexylresorcinol, benzocaine, dextromethorphan, menthol lidocaine, amyl metacresol and 2,4-dichlorobenzyl alcohol or pharmaceutically acceptable salts thereof.

The first liquid composition can comprise up to about 10% NSAID by weight of the composition. Typically, the amount of NSAID is from 1% by weight of the first liquid composition to 6% by weight of the composition. More typically, the amount of NSAID is from 2% by weight to 4% by weight of the first liquid composition.

The first composition is formed by combining the pharmaceutically active agent and a solubilising agent.

The solubilising agent is typically selected from cyclodextrins, or combinations thereof.

The solubilising agent is typically selected from cyclodextrins, or combinations thereof. Other solubilising agents include non-ionic surfactants, such as Tween, and propylene glycol.

The cyclodextrin can be selected from α, β, γ cyclodextrin and derivatives thereof. Cyclodextrins for use in the present invention include the natural cyclodextrins and their derivatives, including the alkylated and hydroxyalkylated derivatives and the branched cyclodextrins. derivatives bearing sugar residues are of special interest. Especially useful herein are the hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α, β, γ-cyclodextrin. Specific cyclodextrin derivatives for use herein include methyl α cyclodextrin, hydroxyethyl α cyclodextrin, hydroxypropyl α cyclodextrin, dihydroxypropyl α cyclodextrin, methyl β cyclodextrin, hydroxyethyl β cyclodextrin, hydroxypropyl β cyclodextrin, dihydroxypropyl β cyclodextrin, methyl γ cyclodextrin, hydroxyethyl γ cyclodextrin, hydroxypropyl γ cyclodextrin and dihydroxypropyl γ cyclodextrin.

Preferably, the cyclodextrin comprises more than one cyclodextrin. More preferably, the cyclodextrin comprises beta cyclodextrin and beta hydroxypropyl cyclodextrin.

The second composition can comprise a combination of a cellulose and an alkali metal carboxyalkylcellulose.

For the avoidance of doubt, by the term ‘NSAID’ as used herein we mean a non-steroidal anti-inflammatory drug in the form of its free acid.

The compositions of the present invention are all aqueous-based compostions.

As used herein, the term ‘pharmaceutically acceptable salts’ describes alkali metal salts (i.e. those elements of Group I of The Periodic Table), especially sodium or potassium; alkaline earth metal salts (i.e. those elements of Group II of The Periodic Table), especially calcium or magnesium; other metal salts, for example aluminium salts; amino acid salts, for example the lysine or arginine salts; or, amine salts, for example meglamine salt.

As used herein, the term “thixotropic” is used to describe one or more agents, e.g., certain gels or viscous liquids, which sheer thin when subjected to vibratory forces like simple shaking, and then thicken again when left standing. Thixotropic behaviour is observed when long-chain molecules tend to orient themselves in the direction of flow; as the applied force is increased, the resistance to flow is decreased. Yet when high shear stress is removed, the solution will quickly revert to its original viscous state.

A sprayable composition in accordance with the present invention is a composition capable of being applied to the mucosal surface of the throat/pharyngeal area in the form of a spray of fine drops.

The terms ‘solubilising agent’, ‘solubilising agents’, ‘solubiliser’ and ‘solubilisers’ have the same meaning.

As used herein, terms such ‘preferably’, ‘more preferably’, ‘most preferably’, ‘typically’, ‘more typically’ and ‘most typically’ indicate preferred features of the compositions of the present invention.

As used herein, ‘about’ means within a range of +/−10% of a given value or number.

Viscosity measurements were taken at room temperature (25° C.) using a Discovery Series Hybrid Rheometer fitted with a 40 mm cross hatched plate measuring system set to a gap of 300 μm during analysis. A solvent trap cover was employed to minimise drying of the sample at the exposed edges. Following a 30 s equilibration time at room temperature (25° C.), the samples were exposed to a shear rate upsweep, 10 s-1 to 15000 s-1, logarithmically scaled, 8 points per decade of shear rate, shear applied for 30 s at each rate with viscosity calculated over the final 5 seconds of each step.

The Yield Point refers to the point of stress at which a product starts to flow and its viscosity drops. The Yield Point was measured using a Discovery Series Hybrid Rheometer fitted with a 40 mm cross hatched plate measuring system set to a gap of 300 μm during analysis. A solvent trap cover was employed to minimise drying of the sample at the exposed edges.

Following a 60 s equilibration time at room temperature (25° C.), the samples were subjected to a shear stress sweep from 0.1 Pa to 100 Pa, logarithmically spaced, 8 points per decade of shear stress.

Steady-state sensing was employed to ensure individual viscosity readings have reached an acceptable degree of elastic or thixotropic equilibrium before being recorded. At each step of the test viscosity was monitored every 10 seconds. Viscosity was recorded only when 3 successive measurements were within 5% of each other. A 60 s timeout was set: if an equilibrium viscosity was not achieved after that time the viscosity at that instant was recorded regardless of degree of equilibrium.

Rheology experiments to determine the recovery of the initial viscosity of the composition were carried out using a Kinexus Rheometer using parallel plates. Measurements were performed at room temperature (25° C.). A three step shear test was performed. In a three step shear test a sample is exposed for 60 s at a low shear rate of 0.1 s⁻¹, followed by a high shear rate of 100 s⁻¹, and finally a low shear rate of 0.1 s⁻¹ for 10 minutes.

The present invention will now be described in more detail with reference to the following Examples and Figures in which:

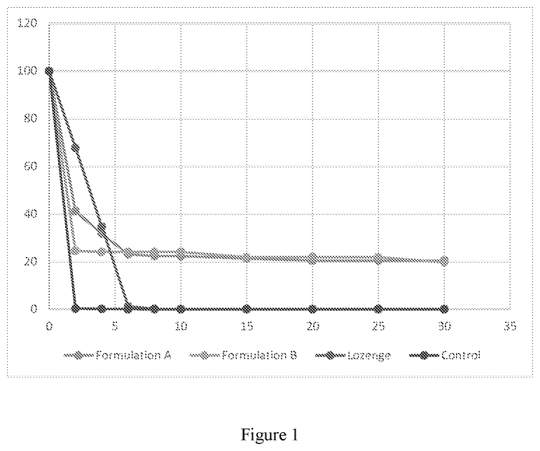

FIG. 1 illustrates the retention profile for example compositions of the present invention, a lozenge and a control composition when the model is at 45°.

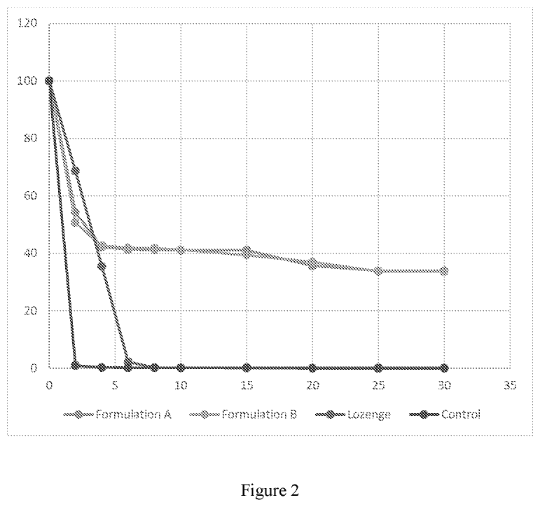

FIG. 2 illustrates the retention profile for example compositions of the present invention, a lozenge and a control composition when the model is at 30°.

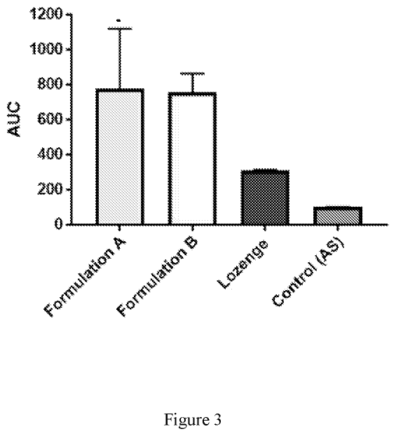

FIG. 3 illustrates the corresponding area under the curve that was calculated based on the retention profile for each composition.

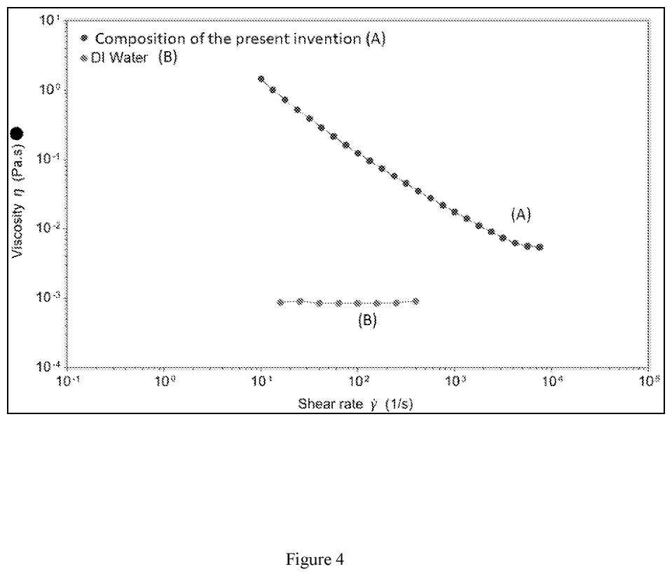

FIG. 4 illustrates the effect of shear rate on the viscosity of an example embodiment of the compositions of the present invention and deionised water.

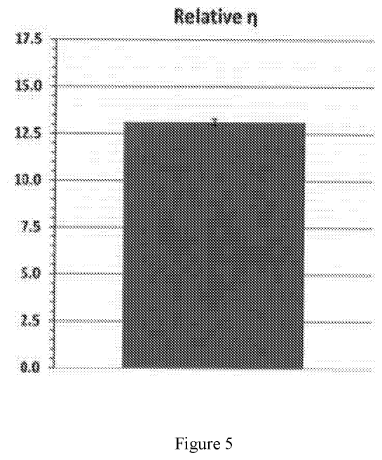

FIG. 5 illustrates the relative mucoadhesive properties of a composition of the present invention when measured at a low shear rate.

| Ingredient | Ex 1 (% wt) | Ex 2 (wt %) | Ex 3 (wt % | Ex 4 (wt %) |
|---|---|---|---|---|
| Purified Water | 85.080 | 86.080 | 86.63782 | 86.83782 |
| Microcrystalline cellulose | 3.115 | 2.225 | 2.225 | 2.047 |
| Carboxymethyl cellulose sodium | 0.385 | 0.275 | 0.275 | 0.253 |
| Beta-cyclodextrin | 4.08 | 4.08 | 3.0440 | 3.04400 |
| Disodium hydrogen phosphate | 3.07 | 3.07 | 2.75400 | 2.75400 |
| Hydroxypropyl Beta Cyclodextrin | 2.15 | 2.15 | 2.39700 | 2.39700 |
| Flurbiprofen | 1.56 | 1.56 | 1.40224 | 1.40224 |
| Sodium Hydroxide | 0.23 | 0.23 | 0.22800 | 0.22800 |
| Citric acid monohydrate | 0.11 | 0.11 | 0.10000 | 0.10000 |
| Methyl paraben | 0.2 | 0.2 | 0.21100 | 0.21100 |
| Propyl paraben | 0.02 | 0.02 | 0.04200 | 0.04200 |
| Sodium Saccharin | — | — | 0.05000 | 0.05000 |
| Xanthan Gum | — | — | 0.2000 | 0.2000 |
| Flavour | — | — | 0.43394 | 0.43394 |
| Total | 100.000 | 100.000 | 100.000 | 100.000 |

Method of Preparation

The composition as described in either Example 1 or Example 2 can be prepared in the following way. An aqueous solution of sodium hydroxide is prepared. Separately, an aqueous mixture of flurbiprofen, disodium hydrogen phosphate, hydroxypropyl beta cyclodextrin, beta-cyclodextrin, citric acid, methyl paraben and propyl paraben was also prepared. The aqueous solution of sodium hydroxide was added to the flurbiprofen-containing aqueous mixture with cooling to form a premix solution. In a separate vessel a combination of microcrystalline cellulose and sodium carboxymethylcellulose are added to water. The mixture is homogenised before the premix solution is added while stirring of the homogenised mixture is maintained. The resulting product is now ready to be dispensed into suitable storage containers.

In Example 3 and Example 4 the xanthan gum is added to the aqueous combination of microcrystalline cellulose and sodium carboxymethylcellulose.

The retention profile of the embodiments of the compositions of the present invention was examined using apparatus which measures adhesion on mucosal tissue. The model comprises a slope to which is applied a cellulose membrane. The cellulose membrane is hydrated in a mucin-containing aqueous mixture and is used to mimic the surface of the throat. A sample of the thixotropic composition of the present invention is applied to the membrane. The membrane is then continuously washed with artificial saliva for 30 mins. Samples are collected at regular intervals. Each eluted sample was tested to determine how much of the composition was retained on the slope.

FIG. 1 illustrates the retention of the composition on the slope of the model when at an angle of 45°. This angle was chosen to simulate the application of the composition to an individual when awake. The results are shown in Table 1.

TABLE 1
Awake Model

| | Formulation A | Formulation B | Lozenge | Example |
|---|---|---|---|---|
| Time (mins) | (% retained) | (% retained) | (% retained) | (% retained) |
| 0 | 100 | 100 | 100 | 100 |
| 2 | 41.1 | 24.52 | 67.64 | 0.27 |
| 4 | 31.7 | 24.02 | 34.66 | 0.16 |
| 6 | 23.06 | 24.02 | 1.23 | 0 |
| 8 | 22.32 | 24.02 | 0.17 | 0 |
| 10 | 22.32 | 24.02 | 0.11 | 0 |
| 15 | 21.25 | 21.85 | 0.11 | 0 |
| 20 | 20.40 | 21.85 | 0.11 | 0 |
| 25 | 20.40 | 21.85 | 0.11 | 0 |
| 30 | 20.40 | 19.68 | 0.11 | 0 |

FIG. 2 illustrates the retention of the composition on the slope of the model when at an angle of 30°. This angle was chosen to simulate the application of the composition to an individual when asleep. The results are shown in Table 2.

TABLE 1
Asleep Model

| | Formulation A | Formulation B | Lozenge | Example |
|---|---|---|---|---|
| Time (mins) | (% retained) | (% retained) | (% retained) | (% retained) |
| 0 | 100 | 100 | 100 | 100 |
| 2 | 54.25 | 50.73 | 68.58 | 1.01 |
| 4 | 42.26 | 42.71 | 35.53 | 0.33 |
| 6 | 41.39 | 41.93 | 2.29 | 0.24 |
| 8 | 41.23 | 41.79 | 0.27 | 0.22 |
| 10 | 41.14 | 41.28 | 0.2 | 0.21 |
| 15 | 41.10 | 39.40 | 0.16 | 0.21 |
| 20 | 35.69 | 37.02 | 0.16 | 0 |
| 25 | 33.97 | 33.72 | 0.16 | 0 |
| 30 | 33.92 | 33.72 | 0.16 | 0 |

As can be seen from FIGS. 1 & 2, the compositions are retained on the slope of the model for a significantly longer time than the lozenge. The flurbiprofen in the compositions is therefore able to provide a longer local effect on the surface of the throat as can be seen from the “Area Under The Curve” plot shown in FIG. 3.

The results obtained using this model suggest that the composition remains on the mucosal tissue despite the action of gravitational forces as a result of its high viscosity due to its gel like nature at rest resulting from its high molecular weight; and also as a result of hydrogen bonding and ionic attraction between the composition and the surface of the mucosa.

In addition, a mucoadhesion study was carried out on a composition in accordance with the present invention. The mucin solution is used to replicate/mimic the mucosal surface of the throat/pharyngeal area. The sample for was prepared as follows:

A mucin solution was made using deionised water and porcine gastric mucin (II). The solution was made to a concentration of 10%, the pH adjusted to 6.2 using 0.5M NaOH solution before being diluted with DI water to a final concentration of 6% before use. 3 g of the prepared mucin solution was mixed with an equal weight of the sample under test, giving a final mucin concentration of 3% (w/w). For control samples, the mucin solutions and samples under investigation were diluted to 50% (w/w) of their initial concentration using deionised water. All samples were prepared in duplicate and allowed to equilibrate overnight at 5° C. before any analysis was conducted.

The mucoadhesion study testing was performed on a research rheometer (DHR2, TA Instruments) fitted with a 60 mm aluminium plate measuring system set to a gap of 200 μm during analysis. A solvent trap cover was employed to minimise drying of the sample at the exposed edges.

The mucoadhesion study was carried out to identify rheological changes that indicate synergistic interactions developed between the compositions of the present invention and mucin solutions. The changes were identified and quantified by measuring viscosity at low and high shear rates.

The example composition was analysed both individually and when mixed with a prepared mucin solution. Quantifying zero shear viscosity (ηₒ) was the ideal aim for the example composition; it is believed that this zero shear viscosity provides a suitable indicator of the behaviour of the product in situ. This was entered into the following two equations to give the “rheological synergism parameters” Δηₒ and Δηₒ/ηₒ.

Δηₒ=ηₒ₍ₘᵢₓ₎−(ηₒ₍ₛₐₘₚₗₑ₎+η_{o(mucin)})

Δηₒ/ηₒ+1, where ηₒ=ηₒ₍ₛₐₘₚₗₑ₎+η_{o(mucin)}

Δηₒ is the difference between the actual viscosity values of the composition mixed with mucin and the theoretical value; the theoretical value is defined as the sum of the ηₒ values of the sample and the mucin when analysed individually. Δηₒ/ηₒ+1 describes the relative rheological synergism, this expresses the relative increase in ηₒ with regards to the sample and mucin alone.

A value greater than one indicates some interaction with mucin; a value of 2 for example would mean the measured viscosity of the sample mixed with mucin is double what was expected.

The results for the composition tested are shown in Table 3.

TABLE 3

| | η | η Mucin | η Mix | Δη | Relative |
|---|---|---|---|---|---|
| Shear Rate | (mPa.s) | (mPa.s) | (mPa.s) | (mPa.s) | η |
| Low Shear (1 s⁻¹) | 1257 | 3.48 | 16564 | 15304 | 13.1 |
| High Shear (100 s⁻¹) | 32.8 | 3.48 | 507 | 471 | 14.0 |

FIG. 5 illustrates the result obtained at low shear rate.

The composition contains several functional groups capable of forming hydrogen bonds. The presence of these groups promotes a strong interaction with the mucosal surface of the throat allowing the composition to remain on the mucosal tissue of the throat for longer. In addition, as the gel matrix resists being readily washed off by saliva as a result of its structure, the attraction force between the composition and the surface of the mucosa has more time to form. This increase the contact time of active with the throat enhancing topical action and efficacy

As can be seen from FIG. 4, when a shear force or stress is applied to the compositions of the present invention the viscosity decreases. A higher rate of shear force/stress results in the composition having a lower viscosity. On removal of the shear force/stress the viscosity of the composition returns to its initial viscosity confirming that the compositions exhibit shear-thinning or thixotropic behaviour.

An advantage of the present invention is that there is provided a composition which has a viscosity that is sufficient to allow it to be retained on the surface of the throat for a sufficient period of time to allow the active contained therein to exhibit a local effect but on application of a shear force can be converted into a sprayable composition that can be applied to the inflamed/infected area on the surface of the throat with a high degree of accuracy.

Further modifications of the invention can be made without departing from the scope of the invention described herein.

1. A composition comprising a pharmaceutically active ingredient, a solubilising agent and a thixotropic agent.

2. The composition as claimed in claim 1, wherein the composition is in the form of a solution.

3. The composition as claimed in claim 1, wherein the composition is in the form of a sprayable composition.

4. The composition as claimed in claim 1, wherein the pharmaceutically active ingredient is selected from the group consisting of NSAIDs and pharmaceutically acceptable salts thereof.

5. The composition as claimed in claim 4, wherein the NSAID is selected from the group consisting of flurbiprofen, ketoprofen or diclofenac.

6. The composition as claimed in claim 4, wherein the NSAID is flurbiprofen.

7. The composition as claimed in claim 1, wherein the composition comprises from 1% to about 5% NSAID by weight of the composition.

8-10. (canceled)

11. The composition as claimed in claim 1, wherein the viscosity of the composition returns to its initial viscosity within 30 seconds following withdrawal of the shearing force.

12-14. (canceled)

15. The composition as claimed in claim 1, wherein the thixotropic agent is selected from combinations of one or more celluloses and one or more alkali metal carboxyalkylcelluloses and is present in an amount of about 1.5 to about 5 w/w %.

16. The composition as claimed in claim 15, wherein the combination of cellulose and alkali metal carboxyalkylcellulose is microcrystalline cellulose and sodium carboxymethylcellulose.

17-18. (canceled)

19. The composition as claimed in claim 1, wherein the solubilising agent is present in the composition in an amount of 3-10 w/w %.

20. (canceled)

21. The composition as claimed in claim 1, wherein the solubilising agent is selected from the group consisting of one or more cyclodextrins.

22-25. (canceled)

26. The composition as claimed in claim 1, wherein the composition further comprises one or more stabilisers present at an amount of 0.05-5% w/w.

27. The composition has claimed in claim 26 wherein the one or more stabilisers is selected from the group consisting of ethyl cellulose, hydroxypropyl methyl cellulose and xanthan gum.

28-31. (canceled)

32. The composition as claimed in claim 1, wherein the pH of the composition is from 6.0-8.0.

33-42. (canceled)

43. A composition comprising:
1-5% an NSAID;
1-5% a combination of a cellulose and an alkali metal carboxyalkylcellulose;
1-15% one or more cyclodextrins;
0.1-10% one or more pH adjusters;
0.1-15% one or more buffers; and
0.05-5% one or more stabilisers.

44-45. (canceled)

46. The composition as claimed in claim 44, wherein the composition comprises:
1-2% Flurbiprofen;
2-4% a combination of a cellulose and an alkali metal carboxyalkylcellulose;
5-7% one or more cyclodextrins;
0.1-5% one or more pH adjusters;
2-4% one or more buffers; and
0.1-3% one or more stabilisers.

47-51. (canceled)

52. The composition as claimed in claim 43, wherein the comprises:
1-2% Flurbiprofen;
2-4% A combination of microcrystalline cellulose and sodium carboxymethylcellulose;
5-7% A combination of beta-cyclodextrin and hydroxypropyl beta cyclodextrin;
0.1-5% Sodium hydroxide;
1-10% Disodium hydrogen phosphate;
0.1-5% Citric acid monohydrate; and
0.1-0.3% Xanthan gum.

53. A thixotropic composition comprising an active pharmaceutical ingredient having a low shear viscosity of 30-80 Pascal seconds (Pa·s) and high shear viscosity of 0.01-5 Pascals seconds (Pa·s).

54-100. (canceled)

101. The composition as claimed in claim 53, wherein the composition is in the form of a solution comprising:
1-5% by weight of flurbiprofen or pharmaceutically acceptable salt thereof,
5-7% by weight of a solubilising agent selected from cyclodextrins or combinations thereof;
1.5-5% by weight of a thixotropic agent selected from combinations of one or more celluloses and one or more alkali metal carboxyalkylcelluloses;
0.1-1% by weight of one or more stabilisers selected from ethyl cellulose, hydroxypropyl methyl cellulose and xanthan gum;
1-5% by weight of one or more buffers selected from citric acid, disodium hydrogen phosphate, sodium hydrogen phosphate and combinations thereof; and
0.1-1% by weight of one or more pH adjusters,
wherein the composition has a low shear viscosity of 50-60 Pascal seconds (Pa·s) and high shear viscosity of 0.1-1 Pascals seconds (Pa·s), and
wherein the composition has a yield point of 1-30 Pa.

102-155. (canceled)
